(19) United States
(12) Patent Application Publication
(10) Pub. No.: US 2009/0311198 A1
(43) Pub. Date: Dec. 17, 2009

(54) Perhydrolase for Tooth Whitening

(76) Inventors: Edward M. Concar, San Francisco, CA (US); Ayrookaran J, Poulose, Belmont, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/224,535

(22) PCT Filed: Feb. 26, 2007
(86) PCT No.: PCT/US2007/005017

§ 371 Date: Jan. 21, 2009

Related U.S. Application Data
(60) Provisional application No. 60/778,999, filed on Mar. 3, 2006.

(51) Int. Cl. A61K 8/66; A61Q 11/00

(52) U.S. Cl. 424/50

(57) ABSTRACT

The present invention provides compositions and methods for the use of perhydrolase to whiten teeth.

### FIELD OF THE INVENTION

The present invention provides compositions and methods for the use of perhydrolase to whiten teeth.

### BACKGROUND OF THE INVENTION

Most people desire to have bright, white teeth, as the teeth and smile are an important part of the overall picture of the face. In contrast, strongly discolored teeth appear unhealthy and/or even repulsive. Whiter teeth tend to be associated with beauty and a healthy lifestyle. In general, people with a brighter smile tend to smile more and are often less self-conscious. In addition, a whiter smile can help minimize the appearance of facial wrinkles, providing a more youthful and energetic appearance. Furthermore, a whiter smile can tend to give a more friendly appearance.

Thus, tooth whitening is of great interest. For many years, few approaches have been used to whiten teeth. Crowns and dentures were long considered the only means for avoiding discoloration due to exposure to antimicrobials (e.g., tetracycline), coffee, wine, tea, and tobacco. Indeed, although tooth bleaching has been used since the 1870s, its use has not been widespread until recent years. Peroxide first came into use for teeth bleaching in the 1880s and remains the most commonly used tooth bleaching method. Newly developed methods include laser treatment.

Although tooth bleaching can be very effective there are some disadvantages to many of the procedures. For example, use of bleach can result in sore gums and/or teeth. In addition, bleaching is not effective for all people. Indeed, even the newer laser methods are not always effective and the length of time that the effect lasts can be relatively short (e.g., 12-18 months). Thus, for some people crowns or veneers remain the best choice. Furthermore, the trays commonly used for home bleaching can be uncomfortable. Indeed, although tooth bleaching has become more commonplace and new methods and compositions have been developed, there remains a need in the art for safe, effective, easy-to-use methods and compositions for tooth whitening.

### SUMMARY OF THE INVENTION

The present invention provides compositions and methods for the use of perhydrolase to whiten teeth. In some embodiments, any suitable peracid finds use in the teeth whitening and/or cleaning methods and/or compositions of the present invention.

The present invention provides oral care compositions comprising at least one perhydrolase enzyme. In some preferred embodiments, the oral compositions are oral care products selected from dentifrices, toothpastes, tooth powders, mouth washes, pre-rinses, teeth whitening products, and denture cleaning agents. In some particularly preferred embodiments, the perhydrolase comprises the amino acid sequence set forth in SEQ ID NO:2. In some alternative preferred embodiments, the perhydrolase is encoded by a DNA sequence comprising the sequence set forth in SEQ ID NO:1. However, it is not intended that the present invention be limited to these specific sequences, as numerous variants, homologues, derivatives, etc., also find use in the present invention, including, but not limited to the enzymes set forth in U.S. patent application Ser. No. 10/581,014 (herein incorporated by reference in its entirety). In some embodiments, the composition comprises an amount of at least one perhydrolase sufficient to whiten teeth. In additional embodiments, the composition further comprises a hydrogen peroxide generating system. In still further embodiments, the composition further comprises hydrogen peroxide. In yet preferred additional embodiments, the composition further comprises a peracid generating system. In some additional preferred embodiments, the composition further comprises an acid selected from peracetic acid and acetic acid.

The present invention also provides methods for bleaching teeth comprising the contacting teeth with the oral care composition comprising a perhydrolase enzyme, under conditions suitable for bleaching teeth. In some preferred embodiments, the oral compositions are oral care products selected from dentifrices, toothpastes, tooth powders, mouth washes, pre-rinses, teeth whitening products, and denture cleaning agents. In some particularly preferred embodiments, the perhydrolase comprises the amino acid sequence set forth in SEQ ID NO:2. In some alternative preferred embodiments, the perhydrolase is encoded by a DNA sequence comprising the sequence set forth in SEQ ID NO:1. However, it is not intended that the present invention be limited to these specific sequences, as numerous variants, homologues, derivatives, etc., also find use in the present invention, including, but not limited to the enzymes set forth in U.S. patent application Ser. No. 10/581,014 (herein incorporated by reference in its entirety). In some embodiments, the composition comprises an amount of at least one perhydrolase sufficient to whiten teeth. In additional embodiments, the composition further comprises a hydrogen peroxide generating system. In still further embodiments, the composition further comprises hydrogen peroxide. In yet preferred additional embodiments, the composition further comprises a peracid generating system. In some additional preferred embodiments, the composition further comprises an acid selected from peracetic acid and acetic acid.

### DESCRIPTION OF THE INVENTION

The majority of people consider clean, white teeth to be aesthetically very desirable. However, human teeth are not truly white, due to inherent yellowness of the teeth (i.e., due to intrinsic staining), and to the accumulation of external stains on the tooth surface (i.e., extrinsic staining). Teeth with noticeable intrinsic and/or extrinsic stains are objectionable to the general public both on the basis of cosmetic appearance and an indication of poor oral hygiene.

Currently, daily toothbrushing with dentifrices is the most commonly used method to remove extrinsic stains. However, toothbrushing alone is incapable of completely preventing the formation of extrinsic stains. Areas of dentition commonly missed during tooth-brushing (e.g., the interproximal tooth surfaces, and the lingual areas of the anterior teeth), are very prone to stain accumulation. Once the stain has formed, it is very difficult to remove without obtaining a professional dental cleaning.

Relatively recently, it was determined that peracetic acid is a surprisingly effective bleaching or whitening agent for discolored or stained human teeth (See e.g., EP 599 435B1 and EP 545 594B1). EP 545 594 indicates that an aqueous 1% (by weight) solution of peracetic acid gives rise to a faster and superior whitening effect when applied to teeth at ambient to oral range temperatures than does a 30% (by weight) aqueous solution of hydrogen peroxide. In addition, peracetic acid can be applied directly to the teeth as by swab application, incorporated in an oral composition such as a toothpaste, gel or rinse that is to be applied topically, or generated in situ in the oral composition by the reaction of a peroxide source such as hydrogen peroxide, urea peroxide, sodium perborate, sodium percarbonate, and metal peroxides, for example, SrO₂, CaO₂ and NaO₂, with a peroxyacid precursor or activator containing labile acetyl groups. Illustrative examples of such activators include tetracetylethylenediamine, pentaacetylglucose, tetracetylglycoluril, sorbitol hexaacetate or fructose pentaacetate.

One of the major disadvantages associated with the use of peracetic acid packaged for home use by the consumer is its relative instability. Dilute 1% aqueous solutions of peracetic acid will substantially decompose in as little as 30 days at ambient temperatures. Storage at 3° C. significantly improves stability, but not to the extent required for the normal market age for a consumer or professional product. In addition, many common adjuvants present in consumer and professional products such as flavorants and other organic materials can rapidly react with peracetic acid, destroying both the adjuvants and the peracetic acid.

These factors tend to dictate that a preferred approach for the employment of peracetic acid chemistry in dentifrice applications is to generate the peracetic acid in situ at the time of use. In these prior embodiments, a source of hydrogen peroxide and a carboxylate derivative of acetic acid, such as an amide or an ester, can be mixed together in water at a pH high enough to generate sufficient concentration of perhydroxyl anion from the hydrogen peroxide. In contrast, the present invention provides an in situ means to enzymatically generate peracid for tooth whitening.

In addition, U.S. Pat. No. 5,055,305 (incorporated herein by reference in its entirety) describes effervescent tablets for the in vitro cleaning of dentures which contain, as essential components, a bleaching agent which comprises salts of persulfate perborate or pyrophosphate hydrates or metal peroxides, a peroxyacid bleach precursor and an effervescence-producing base composition. Among the numerous organic peracid precursors disclosed are carboxylic acid esters such as acetylsalicylic acid (See e.g., BR 836,988; incorporated herein by reference in its entirety). It is contemplated that the in situ enzymatic generation of peracetic acid or other peracids by the perhydrolase of the present invention will also find use in similar denture cleaning methods.

EP 400 858 (incorporated herein by reference in its entirety) describes a granular composition for the in vitro cleaning of dentures comprising an inorganic persalt bleaching agent, an organic peroxyacid bleach precursor and an effervescence generator. It is contemplated that the in situ enzymatic generation of peracetic acid or other peracids by the perhydrolase of the present invention will also find use in similar denture cleaning methods.

In some embodiments, the present invention finds use in the enzymatic generation of peracids from ester substrates and hydrogen peroxide. In some preferred embodiments, the substrates are selected from one or more of the following: formic acid, acetic acid, propionic acid, butyric acid, valeric acid, caproic acid, caprylic acid, nonanoic acid, decanoic acid, dodecanoic acid, myristic acid, palmitic acid, stearic acid, and oleic acid. Importantly, the present invention provides means for effective bleaching/whitening over broad pH and temperature ranges. In some embodiments, the pH range utilized in this generation is 4-10. In some alternative embodiments, the temperature range utilized is between 5° and 40 C. The present invention provides bleaching at the optimum pH of peracid oxidation, as well as providing bleaching at neutral pH, acidic pHs, alkaline pH and at low temperatures.

In those applications where dentifrice compositions are designed for in vivo use, it is essential that the peracetic acid be generated and work quickly, since the user will normally wish to limit the time in which the dentifrice is in contact with the teeth. In addition, the classes of peroxide generators and peroxy acid bleach precursors useful for in vivo application to the teeth is severely limited due to the requirement that these components be physiologically safe and non-irritating to oral tissues. A further requirement for in vivo use is that the peracetic acid is generated at a relatively neutral pH, close to the safe physiological neutral pH of 7.

The in situ generation and use of peracetic acid as provided herein represents various advantages over methods and compositions known in the art, including control over how fast and where the peracetic acid is generated, as well as providing flexibility in choosing a more stable precursor for oral care formulations. This flexibility in enzyme and substrate choice also provides systems that find use in localizing peracid production at the tooth.

During the development of the present invention, various levels of peracetic acid were compared to buffer negative controls, a hydrogen peroxide positive control, and peracetic acid generated by perhydrolase from propylene glycol diacetate and hydrogen peroxide. These results indicated that enzymatically generated peracetic acid produced clinically significant tooth whitening.

In addition, the present invention provides methods and compositions for the stable storage of peracid precursors for teeth whitening applications. These compositions and methods also find us in bleaching of false teeth (e.g., dentures). In addition, in situ generation of peracids using the methods and compositions of the present invention delivers a stronger oxidative species locally for the bleaching and whitening of intrinsic tooth stains, with minimal sensitization of the patient.

Furthermore, the perhydrolase and/or hydrolase enzymes of the present invention are active on various acyl donor substrates, as well as being active at low substrate concentrations, and provide means for efficient perhydrolysis due to the high peracid:acid ratio. Indeed, it has been recognized that higher perhydrolysis to hydrolysis ratios are preferred for bleaching applications (See e.g., U.S. Pat. Nos. 5,352,594, 5,108,457, 5,030,240, 3,974,082, and 5,296,616, all of which are herein incorporated by reference). In some preferred embodiments, the perhydrolase enzymes of the present invention provide perhydrolysis to hydrolysis ratios that are greater than 1. In some particularly preferred embodiments, the perhydrolase enzymes provide a perhydrolysis to hydrolysis ratio greater than 1 and are find use in bleaching.

As indicated above, key components to peracid production by enzymatic perhydrolysis are enzyme, ester substrate, and hydrogen peroxide. Hydrogen peroxide can be either added directly in batch, or generated continuously “in situ.” However, these enzymes also find use with any other suitable source of H₂O₂, including that generated by chemical, electro-chemical, and/or enzymatic means. Examples of chemical sources are the percarbonates and perborates mentioned above, while an example of an electrochemical source is a fuel cell fed oxygen and hydrogen gas, and an enzymatic example includes production of H₂O₂ from the reaction of glucose with glucose oxidase. The following equation provides an example of a coupled system that finds use with the present invention.

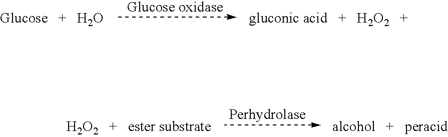

This system generates acid(s) that in some embodiments, results in a lowering of the pH of the system. It is not intended that the present invention be limited to any specific enzyme, as any enzyme that generates H₂O₂ and acid with a suitable substrate finds use in the methods of the present invention. For example, lactate oxidases from Lactobacillus species which are known to create H₂O₂ from lactic acid and oxygen find use with the present invention. Indeed, one advantage of the methods of the present invention is that the generation of acid (e.g., gluconic acid in the above example) reduces the pH of a basic solution to the pH range in which the peracid is most effective in bleaching (i.e., at or below the pKa). Other enzymes (e.g., carbohydrate oxidase, alcohol oxidase, ethylene glycol oxidase, glycerol oxidase, amino acid oxidase, etc.) that can generate hydrogen peroxide also find use with ester substrates in combination with the perhydrolase enzymes of the present invention to generate peracids. Enzymes that generate acid from substrates without the generation of hydrogen peroxide also find use in the present invention. Examples of such enzymes include, but are not limited to esterases, lipases, phospholipases, cutinases, proteases. In some preferred embodiments, the ester substrates are selected from one or more of the following acids: formic acid, acetic acid, propionic acid, butyric acid, valeric acid, caproic acid, caprylic acid, nonanoic acid, decanoic acid, dodecanoic acid, myristic acid, palmitic acid, stearic acid, and oleic acid. Thus, as described herein, the present invention provides definite advantages over the currently used methods and compositions.

Thus, it is contemplated that whiter teeth will be achieved and maintained stain-free through use of the compositions of the present invention. It is contemplated that the perhydrolase of the present invention will find use in compositions such as toothpastes, toothgels, anti-plaque rinses, mouthwashes, etc., to provide and maintain whiter teeth. In some preferred formats, dental rinses comprising the perhydrolase of the present invention are used in order to reach the more inaccessible areas of tooth surfaces, as well as provide penetration into the tooth enamel in order to remove intrinsic stains. It is also contemplated that the present invention will find use in conjunction with regular tooth brushing, although it is not intended that the present invention be limited to the additional use of tooth brushing and/or any other method of dental maintenance.

### DEFINITIONS

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, N.Y. (1991) provide those of skill in the art with a general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the terms “tooth whitening” and “tooth bleaching” are used interchangeably, to refer to improving the brightness (e.g., whitening) of a tooth or teeth. It is intended that the term encompass any method suitable for whitening teeth, including the present invention, as well as chemical treatment, mild acid treatment, abrasive tooth whitening, and laser tooth whitening. In particularly preferred embodiments, the present invention provides a perhydrolase and perhydrolase-containing compositions suitable for whitening teeth.

As used in herein, “intrinsic stains” in teeth refer to the resulting color from chromogens within the enamel and underlying dentin. The intrinsic color of human teeth tends to become more yellow with aging, due to the thinning of the enamel and darkening of the underlying yellow dentin. Removal of intrinsic stain usually requires the use of peroxides or other oxidizing chemicals, which penetrate the enamel and decolorize the internal chromogens.

In contrast to intrinsic stains, “extrinsic stains” form on the surface of the teeth when exogenous chromogenic materials bind to the enamel, usually within the pellicle naturally coating the teeth. Most people accumulate some degree of unsightly extrinsic stains on their teeth over time. This staining process is promoted by such factors as: (1) the ingestion of tannin-containing foods and beverages such as coffee, tea, or red wine; (2) the use of tobacco products; and/or (3) exposure to certain cationic substances (e.g., tin, iron, and chlorhexidine). These substances tend to adhere to the enamel's hydroxyapatite structure, which leads to tooth discoloration and a concomitant reduction in tooth whiteness. Over a period of years, extrinsic stains may penetrate the enamel layer and result in intrinsic stains.

As used herein, the term “perhydrolase” refers to an enzyme that is capable of catalyzing a reaction that results in the formation of sufficiently high amounts of peracid suitable for teeth whitening. In additional preferred embodiments, the perhydrolases of the present invention are characterized by having distinct tertiary structure and primary sequences. In particularly preferred embodiments, the perhydrolases of the present invention comprise distinct primary and tertiary structures. In some particularly preferred embodiments, the perhydrolases of the present invention comprise distinct quaternary structures. In some preferred embodiments, the perhydrolase of the present invention is the M. smegmatis perhydrolase, while in alternative embodiments, the perhydrolase is a variant of this perhydrolase, while in still further embodiments, the perhydrolase is a homolog of this perhydrolase. In further preferred embodiments, a monomeric hydrolase is engineered to produce a multimeric enzyme that has better perhydrolase activity than the monomer. However, it is not intended that the present invention be limited to this specific M. smegmatis perhydrolase, specific variants of this perhydrolase, nor specific homologs of this perhydrolase. In particularly preferred embodiments, the perhydrolase includes those disclosed in US04/40438 and U.S. patent application Ser. No. 10/584,014, as (both of which are incorporated herein by reference in their entirety). In some most particularly preferred embodiments, the perhydrolase comprises the amino acid sequence set forth in SEQ ID NO:2, which in some preferred embodiments is encoded by the DNA sequence set forth in SEQ ID NO:1, both of which are set forth below. However, it is not intended that the present invention be limited to these specific sequences, as variants, homologues and derivatives also find use in the present invention. Indeed, U.S. patent application Ser. No. 10/584,014 describes numerous variants and homologues that find use in the present invention.

(SEQ ID NO: 2)

MAKRILCFGDSLTWGWVPVEDGAPTERFAPDVRWTGVLAQQLGADFEVIE
EGLSARTTNIDDPTDPRLNGASYLPSCLATHLPLDLVIIMLGTNDTKAYF
RRTPLDIALGMSVLVTQVLTSAGGVGTTYPAPKVLVVSPPPLAPMPHPWF
QLIFEGGEQKTTELARVYSALASFMKVPFFDAGSVISTDGVDGIHFTEAN
NRDLGVALAEQVRSLL.

(SEQ ID NO: 1)

5′-ATGGCCAAGCGAATTCTGTGTTTCGGTGATTCCCTGACCTGGGGCTG
GGTCCCCGTCGAAGACGGGGCACCCACCGAGCGGTTCGCCCCCGACGTGC
GCTGGACCGGTGTGCTGGCCCAGCAGCTCGGAGCGGACTTCGAGGTGATC
GAGGAGGGACTGAGCGCGCGCACCACCAACATCGACGACCCCACCGATCC
GCGGCTCAACGGCGCGAGCTACCTGCCGTCGTGCCTCGCGACGCACCTGC
CGCTCGACCTGGTGATCATCATGCTGGGCACCAACGACACCAAGGCCTAC
TTCCGGCGCACCCCGCTCGACATCGCGCTGGGCATGTCGGTGCTCGTCAC
GCAGGTGCTCACCAGCGCGGGCGGCGTCGGCACCACGTACCCGGCACCCA
AGGTGCTGGTGGTCTCGCCGCCACCGCTGGCGCCCATGCCGCACCCCTGG
TTCCAGTTGATCTTCGAGGGCGGCGAGCAGAAGACCACTGAGCTCGCCCG
CGTGTACAGCGCGCTCGCGTCGTTCATGAAGGTGCCGTTCTTCGACGCGG
GTTCGGTGATCAGCACCGACGGCGTCGACGGAATCCACTTCACCGAGGCC
AACAATCGCGATCTCGGGGTGGCCCTCGCGGAACAGGTGCGGAGCCTGCT
GTAA-3′

As used herein, “personal care products” means products used in the cleaning, bleaching and/or disinfecting of hair, skin, scalp, and teeth, including, but not limited to shampoos, body lotions, shower gels, topical moisturizers, toothpaste, toothgels, mouthwashes, mouthrinses, anti-plaque rinses, and/or other topical cleansers. In some particularly preferred embodiments, these products are utilized on humans, while in other embodiments, these products find use with non-human animals (e.g., in veterinary applications).

As used herein, “pharmaceutically-acceptable” means that drugs, medicaments and/or inert ingredients which the term describes are suitable for use in contact with the tissues of humans and other animals without undue toxicity, incompatibility, instability, irritation, allergic response, and the like, commensurate with a reasonable benefit/risk ratio.

As used herein, “cleaning compositions” and “cleaning formulations” refer to compositions that find use in the removal of undesired compounds from teeth (mouthwashes, toothpastes) etc. The term encompasses any materials/compounds selected for the particular type of cleaning composition desired and the form of the product (e.g., liquid, paste, gel, emulsion, granule, or spray composition), as long as the composition is compatible with the perhydrolase and other enzyme(s) used in the composition.

As used herein, “enhanced performance” in a perhydrolase-containing composition is defined as increasing cleaning of bleach-sensitive stains compared to other compositions, as determined using standard methods in the dental art. In particular embodiments, the perhydrolase of the present invention provides enhanced performance in the oxidation and removal of colored stains. In further embodiments, the perhydrolase of the present invention provides enhanced performance in the removal and/or decolorization of stains.

As used herein, the term “compatible,” means that the cleaning composition materials do not reduce the enzymatic activity of the perhydrolase to such an extent that the perhydrolase is not effective as desired during normal use situations. Specific cleaning composition materials are exemplified in detail hereinafter.

As used herein, “effective amount of perhydrolase enzyme” refers to the quantity of perhydrolase enzyme necessary to achieve the enzymatic activity required in the specific application. Such effective amounts are readily ascertained by one of ordinary skill in the art and are based on many factors, such as the particular enzyme variant used, the cleaning application, the specific composition of the cleaning composition, and whether a liquid or non-liquid (e.g., emulsion) composition is required, and the like.

As used herein, “oral cleaning compositions” refers to dentifrices, toothpastes, toothgels, toothpowders, mouthwashes, mouth sprays, mouth gels, chewing gums, lozenges, sachets, tablets, biogels, prophylaxis pastes, dental treatment solutions, and the like. Oral care compositions that find use in conjunction with the perhydrolases of the present invention are well known in the art (See e.g., U.S. Pat. Nos. 5,601,750, 6,379,653, and 5,989,526, all of which are incorporated herein by reference, in their entirety).

As used herein, “acyl” is the general name for organic acid groups, which are the residues of carboxylic acids after removal of the —OH group (e.g., ethanoyl chloride, CH₃CO—Cl, is the acyl chloride formed from ethanoic acid, CH₃COO—H). The names of the individual acyl groups are formed by replacing the “-ic” of the acid by “-yl.”

As used herein, the term “acylation” refers to the chemical transformation which substitutes the acyl (RCO—) group into a molecule, generally for an active hydrogen of an —OH group.

As used herein, the term “transferase” refers to an enzyme that catalyzes the transfer of functional compounds to a range of substrates.

As used herein, “leaving group” refers to the nucleophile which is cleaved from the acyl donor upon substitution by another nucleophile.

As used herein, the term “enzymatic conversion” refers to the modification of a substrate to an intermediate or the modification of an intermediate to an end-product by contacting the substrate or intermediate with an enzyme. In some embodiments, contact is made by directly exposing the substrate or intermediate to the appropriate enzyme. In other embodiments, contacting comprises exposing the substrate or intermediate to an organism that expresses and/or excretes the enzyme, and/or metabolizes the desired substrate and/or intermediate to the desired intermediate and/or end-product, respectively.

As used herein, the phrase, “stability to proteolysis” refers to the ability of a protein (e.g., an enzyme) to withstand proteolysis. It is not intended that the term be limited to the use of any particular protease to assess the stability of a protein.

As used herein, “oxidative stability” refers to the ability of a protein to function under oxidative conditions. In particular, the term refers to the ability of a protein to function in the presence of various concentrations of H₂O₂ and/or peracid. Stability under various oxidative conditions can be measured either by standard procedures known to those in the art and/or by the methods described herein. A substantial change in oxidative stability is evidenced by at least about a 5% or greater increase or decrease (in most embodiments, it is preferably an increase) in the half-life of the enzymatic activity, as compared to the enzymatic activity present in the absence of oxidative compounds.

As used herein, “pH stability” refers to the ability of a protein to function at a particular pH. In general, most enzymes have a finite pH range at which they will function. In addition to enzymes that function in mid-range pHs (i.e., around pH 7), there are enzymes that are capable of working under conditions with very high or very low pHs. Stability at various pHs can be measured either by standard procedures known to those in the art and/or by the methods described herein. A substantial change in pH stability is evidenced by at least about 5% or greater increase or decrease (in most embodiments, it is preferably an increase) in the half-life of the enzymatic activity, as compared to the enzymatic activity at the enzyme's optimum pH. However, it is not intended that the present invention be limited to any pH stability level nor pH range.

As used herein, “thermal stability” refers to the ability of a protein to function at a particular temperature. In general, most enzymes have a finite range of temperatures at which they will function. In addition to enzymes that work in mid-range temperatures (e.g., room temperature), there are enzymes that are capable of working in very high or very low temperatures. Thermal stability can be measured either by known procedures or by the methods described herein. A substantial change in thermal stability is evidenced by at least about 5% or greater increase or decrease (in most embodiments, it is preferably an increase) in the half-life of the catalytic activity of a mutant when exposed to a different temperature (i.e., higher or lower) than optimum temperature for enzymatic activity. However, it is not intended that the present invention be limited to any temperature stability level nor temperature range.

As used herein, the term “chemical stability” refers to the stability of a protein (e.g., an enzyme) towards chemicals that adversely affect its activity. In some embodiments, such chemicals include, but are not limited to hydrogen peroxide, peracids, anionic detergents, cationic detergents, non-ionic detergents, chelants, etc. However, it is not intended that the present invention be limited to any particular chemical stability level nor range of chemical stability.

As used herein, the terms “purified” and “isolated” refer to the removal of contaminants from a sample. For example, perhydrolases are purified by removal of contaminating proteins and other compounds within a solution or preparation that are not perhydrolases. In some embodiments, recombinant perhydrolases are expressed in bacterial or fungal host cells and these recombinant perhydrolases are purified by the removal of other host cell constituents; the percent of recombinant perhydrolase polypeptides is thereby increased in the sample.

As used herein, “protein” refers to any composition comprised of amino acids and recognized as a protein by those of skill in the art. The terms “protein,” “peptide” and polypeptide are used interchangeably herein. Wherein a peptide is a portion of a protein, those skilled in the art understand the use of the term in context.

As used herein, functionally and/or structurally similar proteins are considered to be “related proteins.” In some embodiments, these proteins are derived from a different genus and/or species, including differences between classes of organisms (e.g., a bacterial protein and a fungal protein). In some embodiments, these proteins are derived from a different genus and/or species, including differences between classes of organisms (e.g., a bacterial enzyme and a fungal enzyme). In additional embodiments, related proteins are provided from the same species. Indeed, it is not intended that the present invention be limited to related proteins from any particular source(s). In addition, the term “related proteins” encompasses tertiary structural homologs and primary sequence homologs (e.g., the perhydrolase of the present invention). In further embodiments, the term encompasses proteins that are immunologically cross-reactive.

As used herein, the term “derivative” refers to a protein which is derived from a protein by addition of one or more amino acids to either or both the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is preferably achieved by modifying a DNA sequence which encodes for the native protein, transformation of that DNA sequence into a suitable host, and expression of the modified DNA sequence to form the derivative protein.

Related (and derivative) proteins comprise “variant proteins.” In some preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In some preferred embodiments, the number of different amino acids between variants is between 1 and 10. In some particularly preferred embodiments, related proteins and particularly variant proteins comprise at least 35%, 40%, 45%, 50%, 55%, 60%, 65%, 70%, 75%, 80%, 85%, 90%, 95%, 97%, 98%, or 99% amino acid sequence identity. Additionally, a related protein or a variant protein as used herein, refers to a protein that differs from another related protein or a parent protein in the number of prominent regions. For example, in some embodiments, variant proteins have 1, 2, 3, 4, 5, or 10 corresponding prominent regions that differ from the parent protein.

Several methods are known in the art that are suitable for generating variants of the perhydrolase enzymes of the present invention, including but not limited to site-saturation mutagenesis, scanning mutagenesis, insertional mutagenesis, random mutagenesis, site-directed mutagenesis, and directed-evolution, as well as various other recombinatorial approaches.

As used herein, the term “analogous sequence” refers to a sequence within a protein that provides similar function, tertiary structure, and/or conserved residues as the protein of interest (i.e., typically the original protein of interest). For example, in epitope regions that contain an alpha helix or a beta sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some preferred embodiments, the tertiary structure and/or conserved residues of the amino acids in the protein of interest are located at or near the segment or fragment of interest. Thus, where the segment or fragment of interest contains, for example, an alpha-helix or a beta-sheet structure, the replacement amino acids preferably maintain that specific structure.

As used herein, “homologous protein” refers to a protein (e.g., perhydrolase) that has similar action and/or structure, as a protein of interest (e.g., an perhydrolase from another source). It is not intended that homologs be necessarily related evolutionarily. Thus, it is intended that the term encompass the same or similar enzyme(s) (i.e., in terms of structure and function) obtained from different species. In some preferred embodiments, it is desirable to identify a homolog that has a quaternary, tertiary and/or primary structure similar to the protein of interest, as replacement for the segment or fragment in the protein of interest with an analogous segment from the homolog will reduce the disruptiveness of the change. In some embodiments, homologous proteins have induce similar immunological response(s) as a protein of interest.

As used herein, “homologous genes” refers to at least a pair of genes from different species, which genes correspond to each other and which are identical or very similar to each other. The term encompasses genes that are separated by speciation (i.e., the development of new species) (e.g., orthologous genes), as well as genes that have been separated by genetic duplication (e.g., paralogous genes). These genes encode “homologous proteins.”

As used herein, “ortholog” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “wild-type” and “native” proteins are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring protein may be obtained in accord with the general methods known to those skilled in the art. The methods generally comprise synthesizing labeled probes having putative sequences encoding regions of the protein of interest, preparing genomic libraries from organisms expressing the protein, and screening the libraries for the gene of interest by hybridization to the probes. Positively hybridizing clones are then mapped and sequenced.

The term “recombinant DNA molecule” as used herein refers to a DNA molecule that is comprised of segments of DNA joined together by means of molecular biological techniques.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); HCl (hydrochloric acid); aa (amino acid); bp (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); MgCl₂ (magnesium chloride); NaCl (sodium chloride); OD₄₂₀ (optical density at 420 nm); ABTS (2,2′-azino-di)3-ethylbenzthiazoline-6-sulfonate; substrate for peroxidase); PAGE (polyacrylamide gel electrophoresis); EtOH (ethanol); PBS (phosphate buffered saline [150 mM NaCl, 10 mM sodium phosphate buffer, pH 7.2]); SDS (sodium dodecyl sulfate); Tris (tris(hydroxymethyl)aminomethane); TAED (N,N,N′N′-tetraacetylethylenediamine); w/v (weight to volume); v/v (volume to volume); Per (perhydrolase); per (perhydrolase gene); Ms (M. smegmatis); MS (mass spectroscopy); Amersham (Amersham Life Science, Inc. Arlington Heights, Ill.); ICN (ICN Pharmaceuticals, Inc., Costa Mesa, Calif.); Pierce (Pierce Biotechnology, Rockford, Ill.); Amicon (Amicon, Inc., Beverly, Mass.); ATCC (American Type Culture Collection, Manassas, Va.); Amersham (Amersham Biosciences, Inc., Piscataway, N.J.); Becton Dickinson (Becton Dickinson Labware, Lincoln Park, N.J.); BioRad (BioRad, Richmond, Calif.); Clontech (CLONTECH Laboratories, Palo Alto, Calif.); Difco (Difco Laboratories, Detroit, Mich.); GIBCO BRL or Gibco BRL (Life Technologies, Inc., Gaithersburg, Md.); Sigma (Sigma Chemical Co., St. Louis, Mo.); Sorvall (Sorvall Instruments, a subsidiary of DuPont Co., Biotechnology Systems, Wilmington, Del.); SPSS (SPSS Inc., Chicago, Ill.); Microsoft (Microsoft, Inc., Redmond, Wash.); Agilent (Agilent Technologies, Palo Alto, Calif.); Minolta (Konica Minolta, Ramsey, N.J.); and Continental (Continental Diamond Tool Corp., New Haven, Ind.).

### Tooth Samples

In the following Examples, human permanent teeth with natural intrinsic stains were obtained from oral surgeons. Selection of appropriate teeth was based on the presence of intrinsic stain after removal of exogenous stains using methods known in the art involving use of rubber cup polishing with an aqueous pumice slurry. A VITA® dental shade guide was used as a reference for tooth selection. Only specimens with a shade score of A3 or darker after drying were used in these experiments. The test specimens were comprised of cylinders of human enamel 3 mm in diameter mounted in a 10-mm-diameter black acrylic base. The circular pieces of human enamel were cut using a 3-mm diamond core drill (Continental). In order to cut the enamel, the human teeth were first mounted in 2-cm square blocks of self-curing dental acrylic with the labial surface exposed. Each block was then clamped into a small vise and immersed in a container of water to prevent the enamel from burning during the cutting process. Each immersed tooth was positioned directly under and perpendicular to the diamond core drill bit mounted on the end of an electric drill press. At high speed, the diamond core drill was lowered onto the tooth and gentle pressure was applied as the bit cut through the enamel and dentin. The 3-mm piece of cut enamel was then removed from the end of the diamond core drill and embedded with the aid of a circular mold into black, self-curing dental acrylic, providing circular blocks 10 mm in diameter. This was done in such a manner that the areas of tooth substance exposed by the drilling process were sealed by the acrylic, while the 3-mm surface enamel was left exposed. The finished tooth specimens were examined under a dissecting microscope. Specimens with imperfections were discarded. Acceptable specimens were thoroughly rinsed with distilled water, and refrigerated in a humidor until use.

### Example 1

### Stain Measurement

In these experiments, stain on the teeth was measured using the following method. The amount of the intrinsic stain within the teeth was measured by taking color readings with a Minolta CM-503i Spectrophotometer with diffuse illumination/8° viewing angle and 3 mm aperture. Measurements over the entire visible color spectrum were obtained using the CIELAB (Commission Internationale de L'Eclairage, Recommendations on Uniform Color Spaces. Color Difference Equations. Psychometric color Terms, Suppl. 2 to CIE publication 15 (E-13.1) 1971/(TC-1.3), 1978, Paris: Bureau Central de la CIE, 1978) color scale. This scale quantifies color according to 3 parameters, L* (lightness-darkness value), a* (red-green chroma), and b* (yellow-blue chroma). In order to obtain reproducible readings, the enamel specimens were allowed to air-dry at room temperature for 30 minutes before color measurements were taken. When the specimens were exposed to air, the teeth became whiter as they dry within the first few minutes and the color parameters change dramatically, but stabilized after 30 minutes.

Measurements were obtained by aligning the center of the circular 3-mm segment of enamel directly over the 3-mm-diameter targeting aperture of the Minolta spectrophotometer, which enabled accurate positioning of the specimens for each test. An average of 3 color readings using the L*a*b* scale were taken for each specimen.

### Example 2

### Treatment of Teeth

In this Example, methods of treating the tooth samples are described. Before treatment, the baseline L*a*b* color scores of the tooth specimens were determined and used to stratify the teeth samples into balanced groups of 8 specimens each. For testing, each tooth specimen was immersed in 20 ml of the respective test solution for 30 minutes. The test solutions were stirred at 60 rpm with a mechanical stirrer for the duration of the exposure.

Following each 30-minute treatment period, the specimens were rinsed and then placed back into fresh test solution. Treatments were conducted consecutively with color readings taken after 1, 2, 4, 8, 16, and 24 hours. As a control group, 8 teeth were similarly treated in distilled water.

The stain calculations were made as described below. The difference between the baseline and post-test readings for each color factor (L*, a*, and b*) represents the ability of the test solutions to remove intrinsic stain and thereby whiten the teeth. The data were calculated and defined as follows:
Intrinsic Stain Removed=Color reading after treatment minus baseline tooth color reading.

The overall change in the color of the teeth was calculated using the CIELAB equation ΔE=[(ΔL*)²+(Δa*)²+(Δb*)²]^{1/2}. The individual components of the L*a*b* scale were also analyzed separately, in order to determine the specific changes in the whiteness (L*), red-green color range (a*), and yellow-blue color range (b*).

The statistical significance of the data obtained in each category were determined by analysis of variance. Data were tabulated using a PC computer and EXCEL® (Microsoft) spreadsheet program and analyzed using the SPSS® (SPSS) statistics program. All comparisons were made using a 2-tail test.

The test groups used in these experiments were:

Group 1—0.20% peracetic acid in 100 mmolar phosphate buffer (pH 7.5-0.0).
Group 2—0.10% peracetic acid in 100 mmolar phosphate buffer (pH 7.5-0.0).
Group 3—0.05% peracetic acid in 100 mmolar phosphate buffer (pH 7.5-0.0).
Group 4—0.02% peracetic acid in 100 mmolar phosphate buffer (pH 7.5-0.0).
Group 5—100 mmolar phosphate buffer (vehicle control).
Group 6—3% peroxide (positive control)
Group 7—distilled water (negative control)
Group 8—enzyme, substrate and peroxide in 100 mmolar phosphate buffer (pH 7.5-8.0).
Group 9—substrate and peroxide in 100 mmolar phosphate buffer (pH 7.5-0.0).

Acetic and peracetic acid in peracid stock solution were titrated with NaOH to obtain the pH of the testing groups (pH 7.5-8.0). As needed, NaOH was added before the peracetic acid was added.

The 0.2% peracetic acid stock solution was prepared by adding 0.56 mL 12.5 M NaOH to 100 mL buffer before adding 0.62 ml 32% peracetic acid (containing 40-45% acetic acid). The remaining peracetic acid solutions were prepared by making consecutive serial dilutions of 50 mL peracid solution into 50 mL buffer. As peracid solutions are unstable, they were used within one hour of preparation (i.e., for two consecutive 30 minute treatments). The enzyme used in these experiments was the M. smegmatis perhydrolase described in US04/40438 (incorporated herein by reference in its entirety). The protocol used to prepare the enzyme groups is below:

Reaction Conditions:

100 mM sodium phosphate pH 7.9

0.17% H₂O₂ (50 mM)

0.42% propylene glycol diacetate (25 mM)

+/−4 ppm enzyme (perhydrolase)

Prepare and Mix:

50 mL 200 mM sodium phosphate pH

5.7 mL 3% H₂O₂

420uL 100% propylene glycol diacetate (PGDA)

˜44 mL distilled H₂O (to 100 mL final volume in volumetric flask)

2× Enzyme Dilution:

Add 60 uL 16,000 ppm enzyme (perhydrolase) stock to 60 uL phosphate buffer

Stock enzyme test solution and 2× enzyme dilutions were prepared. Then, 20 ml of test solution were placed into 2 beakers. Tooth specimens were then placed in each test solution and 10 uL of enzyme dilution were added to one 20 mL test solution beaker and mixed by swirling. The shaker agitation and timer were then started. The solutions were prepared in quantities needed for the experiments scheduled for each day, as the solutions were found to be stable for use throughout an entire day (i.e., approximately 8 hours) of experiments.

The peracetic acid concentrations were determined as described below.

### Reagents:

125 mM citric acid adjusted to pH 5 with KOH (citrate buffer)

250 mM KI in water (stored out of direct light)

100 mM ABTS (substrate) in water (divided into 1 mL aliquots in brown Eppendorf tubes and frozen @-80° C.; thawed in 25° C. water bath)

Fresh peracetic acid (PAA; Sigma 32%) was stored at 4° C. Peracetic acid may vary to 35% and change with time. If desired, it can be titrated for high accuracy.

The solutions were prepared by making a working stock of 25 mM KI by dilution of stock in water. Then, 10 mL of 100 mM ABTS and 2 mL 25 mM KI were added per mL of citrate buffer. This solution (“ABTS reagent”) was made up as needed, including in larger volumes as a working stock. It is useful for up to two days, when kept dark at all times and at ambient temperature.

The standard curve was prepared as described below. A working stock of PAA was prepared by diluting 32% 10⁴-fold to 0.4 mM in water (note: stock contained acetic acid). A dilution series was prepared from the 0.4 mM working stock in water (standard concentrations of PAA made up in B(OH)₃, pH 8.5 buffer hydrolyzed with a half-life of 25 minutes). At ambient temperature, 50 to 100 uL of standard PAA dilution were added to 1 mL of the ABTS reagent. The solution was mixed well and incubated at ambient temperature for 3 minutes. The absorbance at 420 nm (or another chosen wavelength) was then measured. It was found that there was no point in the spectrum that can be subtracted for a baseline shift correction, since ABTS absorbs light over the entire wavelength range from the UV into the near infrared.

A timecourse assay was also conducted. The enzyme reaction was set up and then enzyme was added to the mixture. At various time points, 50 to 100 ul aliquots of enzyme reaction mixture were removed and added to 1 mL of ABTS reagent. The absorbance at 420 nm was then observed.

The ABTS assay was found to discriminate well between PAA and H₂O₂. The standard curve runs were are not entirely linear, ranging in molar extinction response from 61.9*10³ to 44*10³ in going from 19 to 0.59 mM, respectively when read at 420 nm (two moles of ABTS oxidized per mole of PAA). If desired, absorbance is read at additional wavelengths with somewhat lower extinction coefficient. Reaction of ABTS with PAA was rapid and completed in about 1.5 minutes. Based on these experiments, the suggested color development time was set at 3 minutes. Samples were read in a consistent manner in order to produce lowest deviation of results. To minimize errors due to apparent non-linearity, absorbance values should be above 0.1 OD₄₂₀. The results are provided in the following Tables.

TOTAL CHANGE (ΔE) IN INTRINSIC STAIN ON TEETH AFTER TOPICAL
TREATMENT WITH DENTAL SOLUTIONS TESTED AT HOURS 1-8

| | Total Change (ΔE) in Intrinsic Stain Scores† | | | |
|---|---|---|---|---|
| Rinse Solution* | Hour 1 | Hour 2 | Hour 4 | Hour 8 |
| 0.20% Peracetic Acid | 5.31 ± 1.75^{a,b,c} | 6.60 ± 2.37^{a} | 7.37 ± 2.85^{a} | 7.54 ± 2.34^{a} |
| 0.10% Peracetic Acid | 5.60 ± 2.69^{a,b} | 5.38 ± 1.75^{a,b} | 6.09 ± 2.52^{a,b} | 6.58 ± 2.94^{a} |
| 0.05% Peracetic Acid | 3.41 ± 2.19^{b,c,d} | 3.83 ± 1.81^{b} | 4.72 ± 1.67^{b} | 5.04 ± 1.72^{a} |
| 0.01% Peracetic Acid | 2.98 ± 2.57^{b,c,d} | 4.06 ± 2.51^{b} | 4.09 ± 2.33^{b} | 5.11 ± 2.71^{a} |
| 0% Peracetic Acid | 1.30 ± 0.55^{d} | 1.13 ± 0.36^{c} | 1.19 ± 0.55^{c} | 1.58 ± 0.70^{b} |
| 3% H₂O₂ | 6.21 ± 3.46^{a} | 7.15 ± 2.96^{a} | 7.28 ± 2.75^{a} | 7.48 ± 2.95^{a} |
| Distilled H₂O | 1.36 ± 0.56^{d} | 1.05 ± .034^{c} | 1.54 ± 0.38^{c} | 1.49 ± 0.97^{b} |
| Enzyme | 2.72 ± 1.26^{c,d} | 3.93 ± 2.00^{b} | 4.54 ± 1.87^{b} | 5.36 ± 1.66^{a} |
| Enzyme Control | 0.52 ± 2.56^{d} | 1.01 ± 0.56^{c} | 1.51 ± 0.56^{c} | 1.57 ± 0.53^{b} |

†Mean score ± standard deviation, n = 8. Values designated with a different letter are statistically different at p < 0.05 based on ANOVA and SNK test. Values designated with the same letter are not statistically different.
*Teeth were treated with 20 ml of solution over 30 minute intervals.

TOTAL CHANGE (ΔE) IN INTRINSIC STAIN ON TEETH AFTER TOPICAL
TREATMENT WITH DENTAL SOLUTIONS TESTED AT HOURS 10-16

| | Total Change (ΔE) in Intrinsic Stain Scores† | | | |
|---|---|---|---|---|
| Rinse Solution* | Hour 10 | Hour 12 | Hour 14 | Hour 16 |
| 0.20% Peracetic Acid | 7.72 ± 3.12^{a} | 8.53 ± 3.65^{a} | 9.08 ± 3.81^{a} | 9.34 ± 4.10^{a} |
| 0.10% Peracetic Acid | 7.05 ± 3.31^{a} | 7.89 ± 3.47^{a} | 8.71 ± 4.66^{a} | 9.08 ± 4.24^{a} |
| 0.05% Peracetic Acid | 6.08 ± 1.25^{a} | 6.55 ± 1.79^{a} | 6.82 ± 1.91^{a} | 7.72 ± 2.15^{a} |
| 0.01% Peracetic Acid | 5.88 ± 2.55^{a} | 6.79 ± 3.23^{a} | 7.13 ± 3.14^{a} | 7.57 ± 3.05^{a} |
| 0% Peracetic Acid | 1.20 ± 0.32^{b} | 1.35 ± 0.31^{b} | 1.41 ± 0.65^{b} | 1.67 ± 0.75^{b} |
| 3% H₂O₂ | 7.77 ± 3.34^{a} | 9.00 ± 4.07^{a} | 9.68 ± 3.85^{a} | 10.68 ± 3.51^{a} |
| Distilled H₂O | 1.34 ± 0.59^{b} | 1.33 ± 0.58^{b} | 0.98 ± 0.49^{b} | 1.18 ± 0.38^{b} |
| Enzyme | 6.31 ± 1.71^{a} | 7.15 ± 1.58^{a} | 7.49 ± 1.72^{a} | 7.66 ± 1.79^{a} |
| Enzyme Control | 1.36 ± 0.83^{b} | 1.59 ± 0.66^{b} | 1.43 ± 0.84^{b} | 1.58 ± 1.06^{b} |

†Mean score ± standard deviation, n = 8. Values designated with a different letter are statistically different at p < 0.05 based on ANOVA and SNK test. Values designated with the same letter are not statistically different.
*Teeth were treated with 20 ml of solution over 30 minute intervals.

As indicated in the above Tables, a significant dose-response was observed for the peracetic acid solutions during the first four hours of treatment, while a numerical dose-response effect was observed up to 16 hours of treatment.

The enzyme treatment was not statistically different from the various placebo/negative control solutions after 1 hour, but there was a significant tooth whitening effect after 2 or more house. Indeed, after 8 hours, the enzyme and all of the peracetic acid solutions tested were statistically equivalent to the hydrogen peroxide positive control and different from the various negative controls/placebo. As the American Dental Association guidelines consider a delta E tooth whitening changes with a magnitude of at least four to be clinically significant, the systems provided by the present invention provide noticeable tooth whitening of human teeth.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Having described the preferred embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative of preferred embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

We claim:

1. An oral care composition comprising at least one perhydrolase enzyme.

2. The oral care composition of claim 1, wherein said composition is an oral care product is selected from dentifrices, toothpastes, tooth powders, mouth washes, pre-rinses, teeth whitening products, and denture cleaning agents.

3. The oral care composition of claim 1, wherein said composition comprises an amount of said at least one perhydrolase sufficient to whiten teeth.

4. The oral care composition of claim 1, wherein said composition further comprises a hydrogen peroxide generating system.

5. The oral care composition of claim 1, wherein said composition further comprises hydrogen peroxide.

6. The oral care composition of claim 1, wherein said composition further comprises a peracid generating system.

7. The oral care composition of claim 1, wherein said composition further comprises an acid selected from peracetic acid and acetic acid.

8. A method for bleaching teeth comprising contacting said teeth with the oral care composition of claim 1, under conditions suitable for bleaching said teeth.

9. The method of claim 8, wherein said oral care composition is an oral care product selected from dentifrices, toothpastes, tooth powders, mouth washes, pre-rinses, teeth whitening products, and denture cleaning agents.

10. The method of claim 8, wherein said oral care composition comprises an amount of said at least one perhydrolase sufficient to whiten teeth.

11. The method of claim 8, wherein said oral care composition further comprises a hydrogen peroxide generating system.

12. The method of claim 8, wherein said oral care composition further comprises hydrogen peroxide.

13. The method of claim 8, wherein said oral care composition further comprises a peracid generating system.

14. The method of claim 8, wherein said oral care composition further comprises an acid selected from peracetic acid and acetic acid.
